(19) United States
(12) Patent Application Publication
(10) Pub. No.: US 2016/0287649 A1
(43) Pub. Date: Oct. 6, 2016

(54) PHARMACEUTICAL COMPOSITION FOR REDUCING BODY FAT

(71) Applicant: BIOCODEX, Gentilly (FR)

(72) Inventors: Patrice D. CANI, Brussels (BE); Amandine EVERARD, Ottignies (BE); Lucie GEURTS, Brussels (BE); Emilie FARGIER, Compiegne (FR); Marc VERLEYE, Remy (FR); Marie-Emmanuelle LE GUERN, Compiegne (FR)

(21) Appl. No.: 15/035,161

(22) PCT Filed: Nov. 7, 2014
(86) PCT No.: PCT/EP2014/074109

§ 371 Date: May 6, 2016

(30) Foreign Application Priority Data
Nov. 8, 2013 (EP) 13306537.5

(51) Int. Cl. A61K 36/064

(52) U.S. Cl. CPC A61K 36/064

(57) ABSTRACT

The present invention relates to a pharmaceutical composition comprising, as active substance, Saccharomyces boulardii yeast cells, for use for reducing body fat in an individual.

### FIELD OF THE INVENTION

The present invention relates to a pharmaceutical composition useful for reducing body fat, in particular in individuals having an excess of body fat.

### BACKGROUND OF THE INVENTION

Excess body fat is constantly increasing in the general population. Thus, the proportion of overweight or obese individuals, respectively defined by a body mass index (BMI) higher than 25 kg/m² and 30 kg/m², increased from 36.7% to 41.6% between 1997 and 2003 in France. Moreover, obesity prevalence was 14.5% in 2009. Besides, excess body fat is a risk factor for many pathological disorders, notably including cardiovascular events, making its therapeutic management all the more urgent.

If the main axes of fight against this epidemic mostly aim at improving diet and increasing physical exercise of individuals with excess body fat, other strategies, in particular of the surgical and medical type are also explored.

Current drug-based strategies mainly aim at reducing energetic intakes, for example by restricting intestinal lipid absorption (orlistat), or by decreasing appetite, either by promoting satiety (sibutramine) or by reducing food intake-associated satisfaction (rimonabant).

However, the limited efficacy of these methods or their side effects—it has thus been reported that rimonabant could cause serious depressive troubles—have notably led to the withdrawal of sibutramine and rimonabant from the market, leaving very limited therapeutic options and making the exploration of other pathways necessary.

Saccharomyces boulardii (Ultra-Levure®) is a particular strain of the yeast Saccharomyces cerevisiae. This probiotic is mainly indicated as an adjunct to rehydration in the treatment of diarrhea. Its usefulness has notably been established in children (Villarruel et al. (2007) Acta Paediatr 96:538-541; Szajewska et al. (2007) Aliment Pharmacol Ther 25:257-264) and for diarrhea associated with the taking of antibiotics (Surawicz et al. (1989) Gastroenterology 96:981-988; Kotowska et al. (2005) Aliment Pharmacol Ther 21:583-590) or with Clostridium difficile infections (Surawicz et al. (2000) Clin Infect Dis 31:1012-1017). Besides, it has been shown that Saccharomyces boulardii is able to prevent or treat weight loss in individuals suffering from chronic inflammatory bowel diseases and this effect is different from its anti-diarrheal effects (EP 1 693 064 B1).

### SUMMARY OF THE INVENTION

The present invention arises from the unexpected finding that the administration of Saccharomyces boulardii to obese mice allowed to reduce the body fat of these mice.

Thus, the present invention relates to a pharmaceutical composition comprising, as active substance, Saccharomyces boulardii yeast cells, for reducing body fat in an individual.

The present invention also relates to Saccharomyces boulardii yeast cells for use for reducing body fat in an individual.

The present invention also relates to the non-therapeutic use of Saccharomyces boulardii yeast cells for reducing body fat in a subject having no excess body fat or for reducing body fat in a subject which is not overweight or obese.

In a particular embodiment of the pharmaceutical composition as defined above, of the yeast cells for their use as defined above and of the use as defined above, Saccharomyces boulardii yeast cells are not associated or combined to superoxide dismutase (SOD) and/or to a probiotic, in particular of the genus Lactobacillus and more particularly of the Lactobacillus rhamnosus species. Besides, in a particular embodiment of the pharmaceutical composition as defined above, of yeast cells for their use as defined above and of the use as defined above, Saccharomyces boulardii yeast cells are not enriched in selenium.

### DETAILED DESCRIPTION OF THE INVENTION

### Individual/Subject

The individual or the subject according to the invention is an animal, preferably a mammal, and more preferably a human, in particular a man, a woman or a child.

As intended herein, the body fat or adipose mass or fat mass represents the mass of all the white adipose tissues of the body of the individual or the subject. The body mass of the individual or the subject according to the invention can be determined using a weighing scale.

Furthermore, it is preferred that the individual has an excess body, in particular in relation to his stature. This excess body fat is characterized with respect to normality and can be estimated by many methods well known to one of skilled in the art, including nuclear magnetic resonance (NMR), by calculating the body mass index (BMI) of the individual (body weight in kg relative to the square of the height in meters) or by measuring the abdominal perimeter of the individual.

Thus, it is particularly preferred that the individual according to the invention is either overweight or obese. According to a usual definition a human individual is considered overweight if his BMI is higher than or equal to 25 kg/m² and less than 30 kg/m² and the individual will be said obese if his BMI is higher than or equal to 30 kg/m². The individual according to the invention may notably present with severe obesity, in particular characterized in human by a BMI higher than or equal to 35 kg/m².

More generally, it is preferred that the individual according to the invention is a human and has a BMI higher than or equal to 25 kg/m², 26 kg/m², 27 kg/m², 28 kg/m², 29 kg/m², 30 kg/m², 31 kg/m², 32 kg/m², 33 kg/m², 34 kg/m², 35 kg/m² or 40 kg/m².

Besides, the individual according to the invention may also have an abdominal obesity, corresponding in particular to a visceral adipose tissue excess. According to a usual definition a male human individual has an abdominal obesity if the abdominal perimeter is higher than or equal to 94 cm, in particular higher than 102 cm and a female human individual has an abdominal obesity if the abdominal perimeter is higher than or equal to 80 cm, in particular higher than 88 cm. The abdominal perimeter measure is well known to one of skilled in the art: abdomen circumference is thus preferably measured midway between the last floating rib and the top of the iliac crest in a standing individual in gentle expiration.

It is particularly preferred that the individual according to the invention is a man and presents with an abdominal perimeter higher than or equal to 90 cm, 91 cm, 92 cm, 93 cm, 94 cm, 95 cm, 96 cm, 97 cm, 98 cm, 99 cm, 100 cm, 101 cm or 102 cm. It is also preferred that the individual according to the invention is a woman and presents with an abdominal perimeter higher than or equal to 75 cm, 76 cm, 77 cm, 78 cm, 79 cm, 80 cm, 81 cm, 82 cm, 83 cm, 84 cm, 85 cm, 86 cm, 87 cm or 88 cm.

It is also preferred that when the individual according to the invention has an excess body fat, in particular an excess abdominal body fat, which can be evidenced by overweightness or obesity, this excess body fat is established, i.e. it is present at the beginning of the treatment by Saccharomyces boulardii yeast cells according to the invention.

It is further preferred that the individual according to the invention suffers from type 2 diabetes and/or metabolic syndrome.

It is also preferred that the individual according to the invention further presents with a hepatic steatosis that Saccharomyces boulardii yeast cells are able to treat. Similarly, it is preferred that the individual according to the invention further presents with a hepatic and/or systemic inflammation that Saccharomyces boulardii yeast cells are able to treat.

Moreover, it is preferred that that administration according to the invention of Saccharomyces boulardii yeast cells to the individual according to the invention does not significantly modify the total amount of bacteria in the gut of the individual. This amount can notably be assessed by quantifying the number of colony forming units (CFU) per g of stool of the individual.

Furthermore, it is preferred, according to the invention, that Saccharomyces boulardii cells allow reducing or decreasing of the body mass of the individual according to the invention, in particular when the individual according to the invention is overweight or obese.

### Non-Therapeutic Use

In the frame of the non-therapeutic use of Saccharomyces boulardii yeast cells for reducing the body fate of a subject according to the invention, the latter does not present with an excess body fat, which means that the subject does not suffer from pathologies associated with an excess body fat or is not considered at risk of developing such pathologies. In a similar way, in the frame of the non-therapeutic use of Saccharomyces boulardii yeast cells for reducing the body mass of a subject according to the invention, the latter not overweight or obese, i.e. the subject does not suffer from pathologies associated with overweightness or obesity or is not considered at risk of developing such pathologies. Accordingly, the reduction of body fat or body mass of the subject is not associated with an improvement of his health status and has no prophylactic virtues. The non-therapeutic use according to the invention is then comparable to a cosmetic use. Preferably, the subject presenting with no excess body fat according to the invention or being neither overweight nor obese according to the invention has thus a BMI less than 25 kg/m² and/or an abdominal perimeter less than 90 or 94 cm if it is a man and less than 80 cm if it is a woman.

### Yeast Cells

As intended herein, the expression “yeast cells” includes viable or dead yeast cells, as a whole or in the form of debris. Preferably, at least some of the yeast cells according to the invention are viable, in particular are viable and cultivable, and more preferably a majority of the Saccharomyces boulardii yeast cell according to the invention are viable, in particular are viable and cultivable. Preferably, the Saccharomyces boulardii yeast cells according to the invention are not under hydrolyzed form. Preferably also, the expression “yeast cells” does not refer to a yeast cell wall composition.

The viability of yeast cells can notably be determined by methylene blue coloration and microscopic observation. The number or viable and cultivable cells, which defines the vitality, can be estimated by determining the number of Colony Forming Units (CFU) in the sample.

By way of example, the number of yeast cell CFU in a liquid sample containing yeasts can be determined by spreading a specified volume of the sample on a solid medium, for example a gel medium, allowing the yeasts to grow, and incubating the solid medium for a period of time, for example 48 h, and at a temperature, for example 30° C., allowing the growth of yeast colonies. The number of colonies relative to the volume spread on the solid medium makes it possible to determine the number of CFU contained in the sample. A detailed protocol for determination of CFU in accordance with the invention is notably described in Toothaker and Elmer (1984) Antimicrobial Agents and Chemotherapy 26:552-556 in the paragraph “Assay for S. boulardii”. Moreover, when the yeast sample is in the form of a solid, for example a lyophilized powder, it is preferred to determine the number of CFU contained in the sample after solvating a specified mass of the sample in an aqueous solution, notably distilled water or a 0.9% NaCl solution at pH 7.

Thus, preferably, the Saccharomyces boulardii yeast cells according to the invention comprise about 10⁸ UFC/g to 10¹² UFC/g, more preferably about 2.10⁹ UFC/g to 2.10¹¹ UFC/g.

A “yeast” according to the invention is a fungus, preferably unicellular. The yeast cells according to the invention are of the species Saccharomyces boulardii. Saccharomyces boulardii is well known to one of skilled in the art and is particularly described in Hennequin et al. (2001) J. Clin. Microbiol. 39:551-559.

Particularly preferably, the cells of Saccharomyces boulardii according to the invention are obtained from medicinal products of the brand Ultra-Levure®, Bioflor®, Codex®, Econorm®, Enflor®, Enterol®, Florastor®, Floratil®, Florestor®, Inteflora®, Perenterol®, Perenteryl®, Precosa®, Reflor®, or Ultra-Levura®, or from deposits in the American Type Culture Collection (ATCC, USA) under reference 74012 or in the Collection Nationale de Culture et de Microorganismes (CNCM, France) under reference I-745.

Preferably also, Saccharomyces boulardii yeast cells according to the invention are lyophilized, such as the Saccharomyces boulardii yeast cells of the brand Ultra-Levure®, Bioflor®, Codex®, Econorm®, Enflor®, Enterol®, Florastor®, Floratil®, Florestor®, Inteflora®, Perenterol®, Perenteryl®, Precosa®, Reflor® or Ultra-Levura®.

Advantageously, the viability and vitality of yeast cells obtained from lyophilizates are greater than what can be obtained with other methods of preservation of yeast cells.

As intended herein, “lyophilization” is a method of preservation in which the yeast cells are frozen and are then submitted to sublimation of the frozen water that they contain to yield a lyophilizate in the form of dry yeast powder preferably containing less than 2% of water and more preferably less than 1% of water. Preferably, the lyophilized yeast cells are obtained from yeast cell concentrates. Any type of method of lyophilization of yeast cells known by a person skilled in the art can be used. However, the yeast cells are preferably lyophilized according to the invention by means of the following method of lyophilization:
cultivate the yeast cells in a liquid nutrient medium until the cells reach a stationary phase;
concentrate the cultivated yeast cells and freeze the concentrate;
lyophilize the concentrate.

Preferably, the Saccharomyces boulardii yeast cells are administered by the oral route. Thus, Saccharomyces boulardii yeast cells according to the invention are preferably in the form of capsules or sachets.

Also preferably, the Saccharomyces boulardii yeast cells according to the invention are administered at a dose of from 2.5·10⁶ to 5·10¹² CFU/kg/d or a dose of from 0.00125 g/kg/d to 25 g/kg/d.

Besides, the pharmaceutical composition as defined above preferably comprises a dose of from 50 mg to 1000 mg, in particular of from 50 mg to 250 mg, of Saccharomyces boulardii yeast cells. In addition, the Saccharomyces boulardii yeast cells for their use as defined above, or for the non-therapeutic use as defined above, are preferably administered at a unit dose of from 50 mg to 1000 mg, in particular of from 50 mg to 250 mg.

As one of skilled in the art will understood, when the amount of yeast cells to be administered is expressed in a mass unit (mg or g) the yeast cells are preferably lyophilized.

Besides, the Saccharomyces boulardii yeast cells can be associated or combined to one or more pharmaceutically acceptable vehicles or excipients.

### DESCRIPTION OF THE FIGURES

FIG. 1A, FIG. 1B, FIG. 1C, FIG. 1D and FIG. 1E

FIGS. 1A-1E respectively represent the body weight, the total body fat (measured by nuclear magnetic resonance), the mesenteric fat mass, the epididymal fat mass and the subcutaneous fat mass in gram (g) of obese diabetic control mice (white column, db-CT) or obese diabetic mice treated with an oral administration of Saccharomyces boulardii yeast cells (black column, db-Sb). The * symbol represents a significant difference (P<0.05) of treated mice with respect to control mice.

FIG. 2

FIG. 2 represents the weight of the liver in gram (g) of obese diabetic control mice (white column, db-CT) or obese diabetic mice treated with an oral administration of Saccharomyces boulardii yeast cells (black column, db-SB). The * symbol represents a significant difference (P<0.05) of treated mice with respect to control mice.

FIG. 3A, FIG. 3B, FIG. 3C and FIG. 3D

FIGS. 3A-3D respectively represent the amount of mRNA of CD11c, MCP-1, IL-1β and F4/80 in the liver of obese diabetic control mice (white column, db-CT) or obese diabetic mice treated with an oral administration of Saccharomyces boulardii yeast cells (black column, db-SB). The * symbol represents a significant difference (P<0.05) of treated mice with respect to control mice.

FIG. 4

FIG. 4 represents the lipid mass comprised in the liver (in mg of whole liver) of obese diabetic control mice (white column, db-CT) or obese diabetic mice treated with an oral administration of Saccharomyces boulardii yeast cells (black column, db-SB). The * symbol represents a significant difference (P<0.05) of treated mice with respect to control mice.

FIGS. 5A, 5B and 5C

FIGS. 5A-5C respectively represent the plasmatic amount of IL-6, IL-1β and TNF-α of obese diabetic control mice (white column, db-CT) or obese diabetic mice treated with an oral administration of Saccharomyces boulardii yeast cells (black column, db-SB). The * symbol represents a significant difference (P<0.05) of treated mice with respect to control mice.

FIG. 6

FIG. 6 represents the mRNA amount of CD11c in the colon of obese diabetic control mice (white column, db-CT) or obese diabetic mice treated with an oral administration of Saccharomyces boulardii yeast cells (black column, db-SB). The * symbol represents a significant difference (P<0.05) of treated mice with respect to control mice.

FIG. 7A, FIG. 7B, FIG. 7C, FIG. 7D, FIG. 7E and FIG. 7F

FIGS. 7A-7F respectively represent the amount of total bacteria and of Bifidobacterium spp, Bacteroides spp, Roseburia spp, Lactobacillus spp. and Akkermansia muciniphila bacteria in the caecum of obese diabetic control mice (white column, db-CT) or obese diabetic mice treated with an oral administration of Saccharomyces boulardii yeast cells (black column, db-SB) expressed as the log₁₀ of the amount of bacteria per gram of caecal contents. The * symbol represents a significant difference (P<0.05) of treated mice with respect to control mice.

### EXAMPLE

### 1. Experimental Protocol

30 genetically obese and type 2 diabetic mice db/db (BKS.Cg-Dock7m+/+Lep db/J) (Charles river) aged 6 weeks were daily fed, by oral gavage, with Saccharomyces boulardii yeast (Ultralevure, Biocodex) (3 g/kg) (db-Sb) or the vehicle (NaCl 0.9%) (db-CT) (n=15 mice per group).

Different parameters were tested during the experiment.

a) Body weight
b) Weight of different tissues and organs after taking: liver, colon, and various adipose tissues (visceral (mesenteric), epididymal and subcutaneous).

Tissues were taken at the end of the experiment, mice were then fastened 5 hours before sacrifice. Mice were first anesthetized with isoflurane (Isoba, Schering-Plough Animal Health, Uxbridge, Middlesex, United Kingdom), blood was then taken from the vena cava and the portal vein, and were eventually euthanized by cervical dislocation. Various tissues and organs were removed: liver, colon and various adipose tissues (visceral (mesenteric), epididymal and subcutaneous).

### c) Body fat

The body fat was measured by NMR (time-domain nuclear magnetic resonance (TD-NMR)) at the end of the experiment (Bruker Minispec mq 7.5 NMR Analyzer, The Bruker Corporation, Billerica, Mass., USA).

d) Several organs were analysed (by qPCR) to study various macrophage inflammatory and infiltration markers (CD11c, IL-1β, F4/80, MCP-1).

For the colon, the subcutaneous adipose tissue and the mesenteric adipose tissue, a part of the tissue or the organ was immersed in a RNA later solution (Applied Biosystems, California, USA) for one week at 4° C. to preserve the quality of RNA. After this treatment, tissues and organs were stored at −80° C. The remainder of the tissues and organs was frozen in liquid nitrogen to be finally stored in a freezer at −80° C.

Tissue RNA was extracted with a tripure solution (Tripure Isolation Reagent, Roche, Mannheim, Suisse) after homogenization using a Tissue Lyser (Qiagen, Hilden, Germany) at 30 Hz for 4 minutes. A 10-minutes centrifugation at 4° C. and 12 000 g allowed removing tissue debris. Integrity and concentration of extracted RNA were measured using a bioanalyser with the RNA 6000 Nano kit (Agilent Technologies, Santa Clara, USA). Integrity of RNA extracted from tissues and organs was measured using the RIN (RNA Integrity Number). Then, a reverse transcription was performed using 1 μg of extracted RNA (Reverse Transcription System, Promega Corporation, Madison, Misconsin, USA). Quantitative PCRs were performed using the MESA FAST qPCR SYBR Green kit (Eurogentec, Seraing, Belgium) in microplates. Microplates were analysed in real-time using a qPCR apparatus of the brand StepOnePlus (Applied Biosystems, Foster city, California, USA). Amplicon amounts of the various macrophage inflammation and infiltration marker genes (CD11c, IL-1β, F4/80, MCP-1) were then reported to the amplicon amount of a control gene encoding the ribosomal protein L19 or RPL19.

e) Total lipid content of the liver

The total lipid content of the liver was quantified by gravimetry after lipid extraction according to the method of Folch (Folch et al. (1957) J. Biol. Chem. 226:497) with a solution containing chloroform and methanol in a 2:1 ratio (Foch solution).

f) The intestinal microbiota was analyzed by qPCR after DNA extraction (QIAamp-DNA stool mini kit (Qiagen; Hilden; Germany)) for the following taxa:
a. Total bacteria.
b. Bifidobacterium spp.
c. Lactobacillus spp.
d. Bacteroides/Prevotella spp.
e. Akkermansia muciniphila.
f. Roseburia spp.

Each taxon was quantified on the basis of a straight-line calibration curve containing a precise quantity of DNA and corresponding to a number of cultivated and quantified cells.

g) The measure of the plasmatic concentration of cytokines IL-1β, TNFα, and IL-6 was performed using the Luminex technology (BioPlex 200, BioRad) using a kit synthetized on demand by Millipore (Milliplex, Millipore, Belgium).

Data statistical analysis of was performed using a Student test with a significance threshold of P<0.05.

### 2. Results

### 2.1. Body Weight, NMR Measured Body Fat, and Weight of Various Adipose Tissues

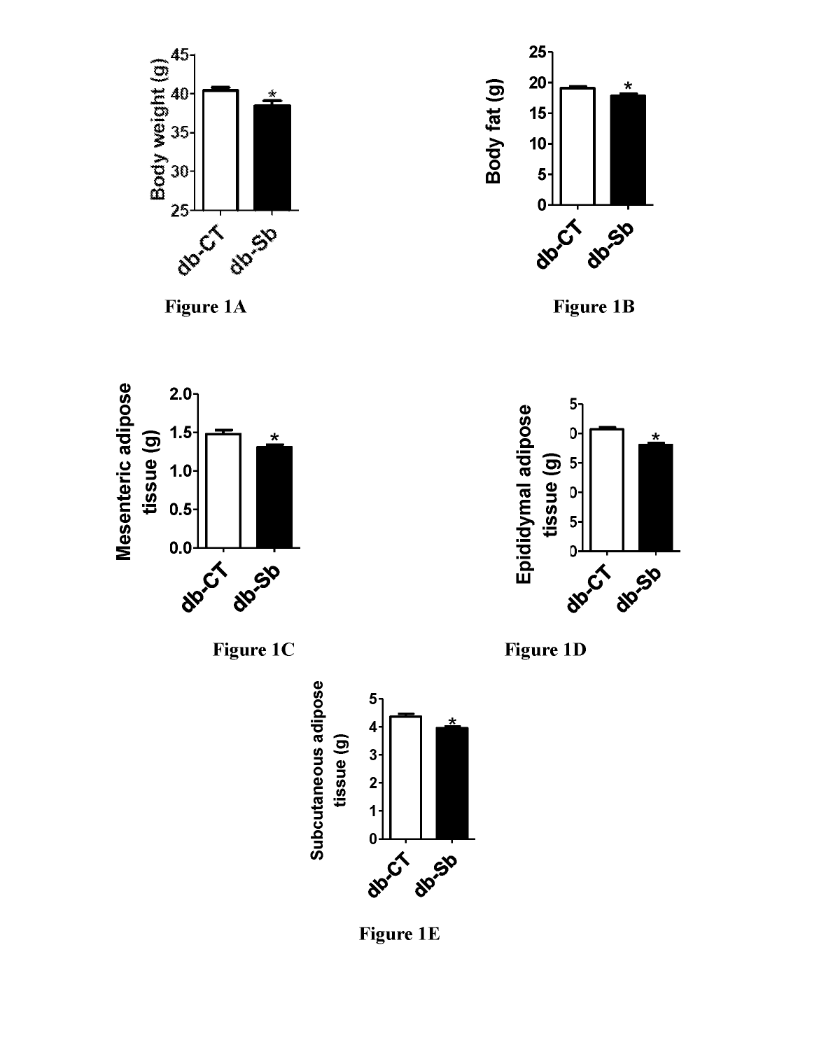

Treatment by S. Boulardii significantly decreases body weight (FIG. 1A), fat accumulation measured by nuclear magnetic resonance (FIG. 1B) and white adipose tissues (FIGS. 1C, 1D, 1E).

### 2.2. Organ Weight at Sacrifice

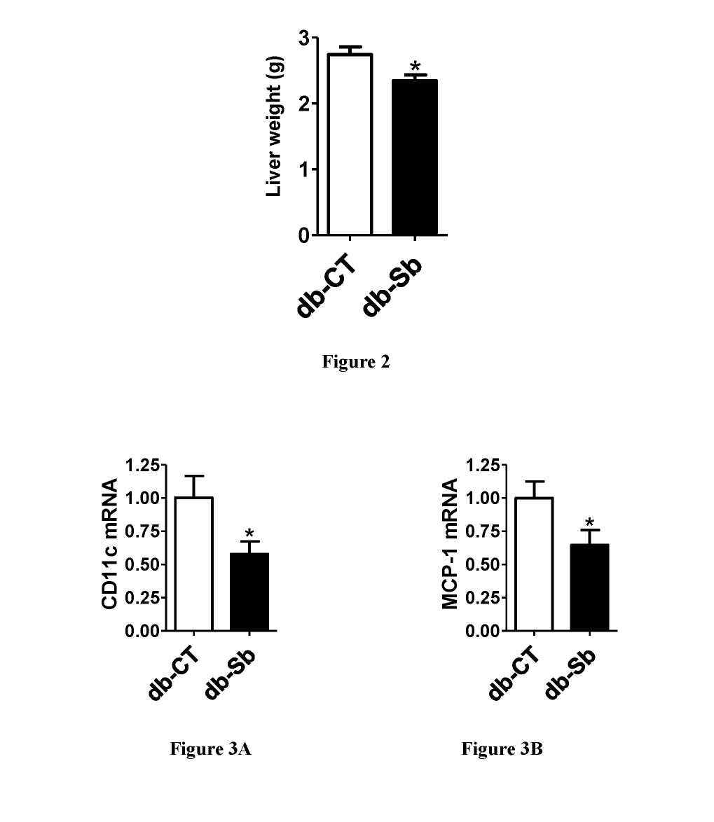

Treatment by S. Boulardii significantly decreases liver weight (−15%) (FIG. 2).

### 2.3. Liver Inflammatory Markers

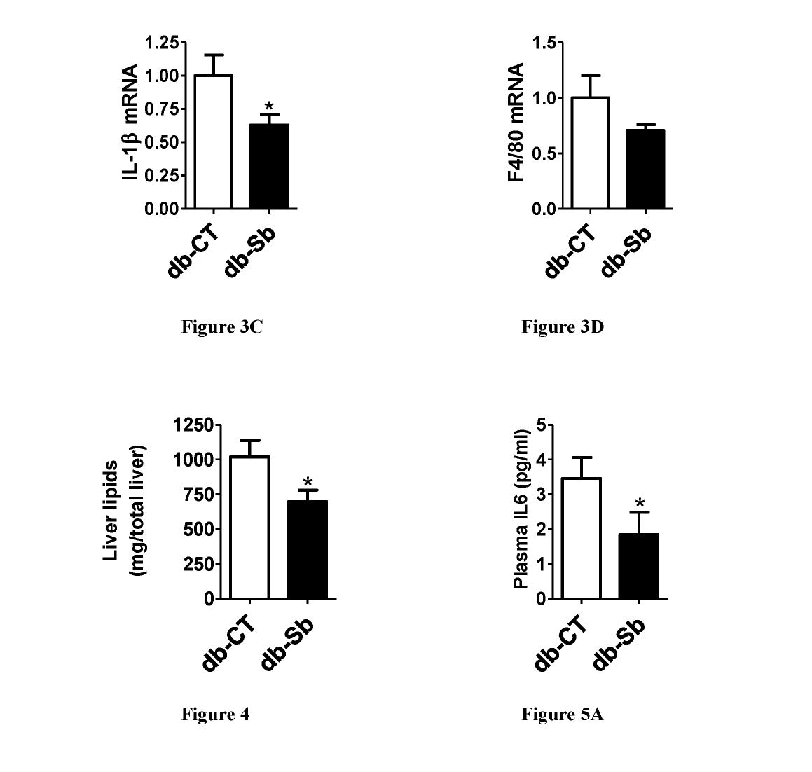

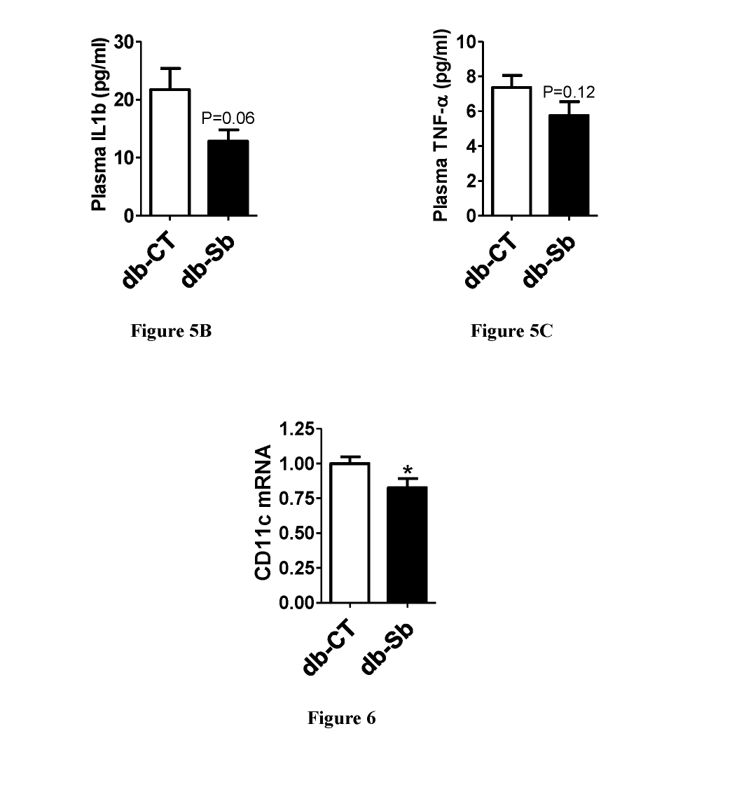

Treatment by S. Boulardii significantly decreases macrophage infiltration, as shown by a decrease in the mRNA expression levels of macrophage infiltration and recruitment markers CD11c, MCP-1, F4/80 (FIGS. 3A, 3B, and 3D), as well as liver inflammation, as shown by a decrease in the mRNA expression level of the β chain the inflammatory cytokine IL-1 (FIG. 3C).

### 2.4. Liver Lipid Content

Treatment by S. Boulardii decreases the total lipid content of liver by 30% (FIG. 4).

### 2.5. Circulatory Cytokines

Various plasmatic cytokines were measured in vena cava plasma using the Luminex technology. Treatment by S. Boulardii induces an important modification of the circulatory immune parameters. Indeed, the plasmatic levels of inflammatory interleukin IL-6 are significantly decreased, besides two other cytokines considered as “pro-inflammatory” (IL-1β and TNFα) tend to be decreased.

### 2.6. Colon

The treatment significantly decreases colonic macrophage infiltration (FIG. 6).

### 2.7. Intestinal Microbiota Analysis

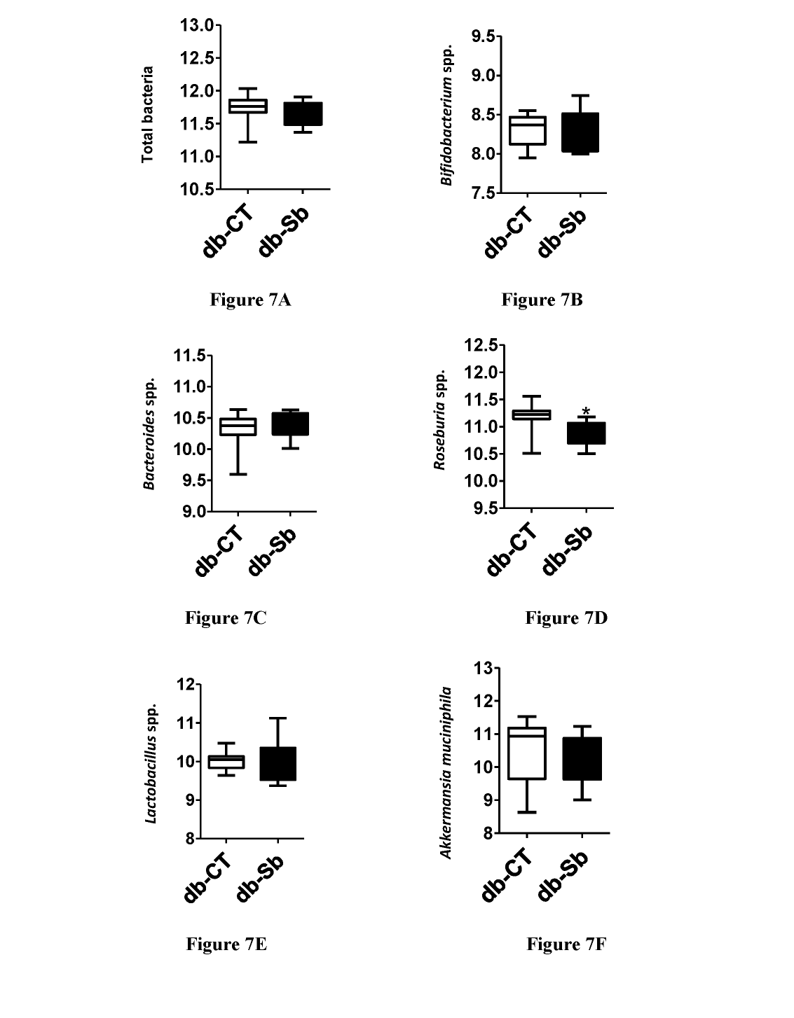

Total bacteria were quantified by qPCR (FIG. 7A) as well as various bacterial genus known to have a potential impact on carbohydrate, lipid metabolism and on the intestinal barrier function, i.e. Bifidobacterium spp. (FIG. 7B), Bacteroides spp. (FIG. 7C), Roseburia spp. (FIG. 7D), Lactobacillus spp. (FIG. 7E) and Akkermansia muciniphila (FIG. 7F).

Administration of S. Boulardii does not significantly modify the total amount of bacteria in the intestine. Indded, there is no significant modification of the bacteria of various investigated taxa except for Roseburia spp (FIG. 7D).

This suggests that S. Boulardii would have a direct impact on host metabolism and potentially not requiring the intervention of other bacteria.

### GENERAL CONCLUSION

Obese and diabetic mice treated with S. Boulardii showed a significant decrease of body fat (reduced by about 10%). This reduction in body fat contributes a body weight decrease (about 5%). Besides, administration of S. Boulardii, does not lead to significant changes in the composition of the intestinal microbiota (total number of intestinal bacteria). In addition, administration of S. Boulardii is associated with a significant decrease of liver weight probably explained by the very clear decrease of the lipid content of this organ (decrease of hepatic steatosis by 30%). Interestingly, all of these effects are associated with a reduction of inflammatory markers (CD11c, MPC-1, IL-1β) in liver tissue. This liver inflammation reduction is also associated with a decrease of various systemic inflammatory parameters. As a conclusion, administration of S. Boulardii significantly decreases body fat and hepatic steatosis in obese and type 2 diabetic mice (db/db), as well as the liver and systemic inflammation.

1. A method for reducing body fat in an individual, comprising administering an effective amount of a pharmaceutical composition comprising, as active substance, Saccharomyces boulardii yeast cells to the individual.

2. The method according to claim 1, wherein the individual has an excess body fat.

3. The method according to claim 1, wherein the individual is obese.

4. The method according to claim 1, wherein the individual presents with abdominal obesity.

5. The method according to claim 1, wherein the individual suffers from type 2 diabetes.

6. The method according to claim 1, wherein the individual suffers from a metabolic syndrome.

7. The method according to claim 1, for further treating hepatic steatosis in the individual.

8. The method according to claim 1, for further treating a liver and/or systemic inflammation in the individual.

9. The method according to claim 1, for further reducing the body weight of the individual.

10. The method according to claim 1, wherein yeast cells are lyophilized.

11. A method for reducing body fat in an individual, comprising administering an effective amount of Saccharomyces boulardii yeast cells to the individual.

12. The method according to claim 11, wherein the individual has excess body fat.

13. The method according to claim 11, wherein the individual is obese.

14. (canceled)

15. (canceled)

16. The method according to claim 11, wherein the individual presents with abdominal obesity.

17. The method according to claim 11, wherein the individual suffers from type 2 diabetes.

18. The method according to claim 11, wherein the individual suffers from a metabolic syndrome.
